(19) United States
(12) Patent Application Publication
(10) Pub. No.: US 2009/0312638 A1
(43) Pub. Date: Dec. 17, 2009

(54) MEDICAL DIAGNOSTIC DEVICE

(75) Inventor: Stewart Gavin Bartlett, Torrensville (AU)

Correspondence Address: Kevin McNamara;IP Manager, Box 736, Torrensville, SA, 5031, omitted

(73) Assignee: SIGNOSTICS PTY LTD, Torrensville, SA (AU)

(21) Appl. No.: 12/306,525

(22) PCT Filed: Jul. 16, 2007
(86) PCT No.: PCT/AU07/00983

§ 371 Date: Dec. 23, 2008

(30) Foreign Application Priority Data
Jul. 17, 2006 (AU) 2006903838

(51) Int. Cl. A61B 8/14

(52) U.S. Cl. 600/443

(57) ABSTRACT

An ultrasound measurement system including a handheld display and processing means, an ultrasound transducer, a processing means of a substantially similar weight to the handheld display and processing means, and a transmission cable interconnecting the handheld display and processing means with the ultrasound transducer and processing means, the cable being of sufficient length to provide a means to mechanically locate the system around the neck of a user.

### FIELD OF THE INVENTION

The present invention relates to medical diagnostic devices and, in particular, to hand-held medical diagnostic devices having processing functionality. The functional field encompasses that of a variety of medical diagnostic devices including but not limited to audio devices, ultrasound scanners, otoscopes, ophthalmoscopes, blood testing devices, endoscopes, electro cardiogram devices, skin lesion testing devices, and vital signs testing devices.

### BACKGROUND OF THE INVENTION

Medical and veterinary practitioners often need to perform numerous tests and procedures on a patient to diagnose illness. The diagnosis of illness usually involves several stages. The first stage is a series of questions and simple diagnostic tests. This stage is relatively inexpensive to perform, and is performed at the patient bedside or in a general/family practice office. If the physician suspects a problem, is unsure, or needs further information, a second stage of test is performed which could include ultrasound imaging, magnetic resonance imaging (MRI), X-Ray, or Computer Aided Tomography. These tests are more expensive, but are still non-invasive. A third stage of tests can be performed including using catheters to inject imaging substances into a patient for clearer images (X-Ray, MRI, CAT, Ultrasound). A fourth stage would be exploratory surgery.

The accuracy and ability of physicians in the first stage of testing has a significant impact on the overall efficiency of a health system. Unnecessary referral for further tests results in waste and unnecessary expense. The first stage of diagnoses includes but is not limited to auscultation, pulse detection, ear and eye inspection, blood pressure detection, visual inspection, temperature detection, neurological tests, and percussion. These tests are carried out using either separate devices or with fingers, hands, eyes, and ears. Some diagnoses require a detailed process of individual tests with the combination of results providing disease indicators.

Devices a physician uses during preliminary examination include stethoscopes, otoscopes, ophthalmoscopes, thermometers, pressure detectors, and neurological kits. Other procedures include palpating to detect arterial pulses, glucose testing, percussing (tapping and listening to the sounds character) and palpation to detect sub-dermal structure, and visual inspection for examining jugular venous pressure and characteristics.

All of these devices, when portable, must be carried and stored individually. Many now include electronic or electrical features and these then require battery power and generally separate battery chargers for each device. When the devices are not portable, or not easily carried, the difficulty of bringing them to the patient may lead to such devices not being used in the first instance, contributing to unnecessary further testing.

There are advantages in cost and patient care in facilitating performance of diagnostic tasks. The ability of physicians to easily record data and images during their investigation is also of great use for reference and monitoring of certain conditions. Under certain circumstances, the ability of physicians to communicate audio or visual data to a colleague at a remote location while maintaining voice contact with the colleague is of great benefit.

The background art contains numerous stethoscope devices for auscultation, including several electronic versions. The first electronic stethoscopes appeared around the same time as the transistor (U.S. Pat. No. 3,182,129), and numerous adaptations have appeared since (U.S. Pat. No. 4,170,717, U.S. Pat. No. 4,598,417, U.S. Pat. No. 6,134,331). Some background art has included interfaces to other devices to allow for telemedicine or further diagnostics, such as devices manufactured by Stethographics, American Telecare Inc, and Cardionics Inc. Other manufacturers have included some additional functionality by clip-on modules, such as the Stethodop covered by U.S. Pat. No. 5,960,089, U.S. Pat. No. 6,106,472 also discloses an ultrasound stethoscope. All of these devices are single function, and can not be configured to perform alternative diagnostic procedures.

Ultrasound systems have traditionally been large bulky devices. Recent developments have seen some portable ultrasound devices produced by manufacturers such as Sonosite Inc (U.S. Pat. No. 5,722,412 and U.S. Pat. No 6,126,608), Terason Inc (U.S. Pat. No. 6,106,472), and Pie Medical (U.S. Pat. No. 6,126,608). These devices are dedicated ultrasound devices, do not implement alternative diagnostic functions, and are not of weight or size to be easily carried by a physician.

Single function otoscope and ophthalmoscope type devices have been used widely in the field for many years. More recently, single function digital otoscope devices with encapsulated camera have been developed (U.S. Pat. No. 6,626,825). These devices are independent of other devices carried by the physician, requiring their own battery packs, recharging supplies, and carry cases.

Several sensor/processor combinations have been developed. Medtronics (U.S. Pat. No. 6,641,533) and Bayer (U.S. Pat. No. 6,604,050) have background art whereby a sensor system is interfaced to a processing system such as a personal data assistant (PDA). These systems are limited in their flexibility, and usually support one or a small number of applications. PDA's have found widespread use in medical communities and provide a number of different interfaces for attaching external devices. The interfaces include compact flash (CF), Secure Data (SD/SDIO), and Universal Serial Bus (USB). None of the interfaces provided are suitable for medical use as they are not robust enough, and do not provide power efficient means to connect to both low speed interfaces and high speed interfaces.

Currently, physicians use a small number of separate portable devices and manual techniques to evaluate and diagnose patients. These devices include stethoscopes, thermometers, blood pressure cuffs, percussion, and visual inspection. Portable ultrasound devices have also been recently developed, although these remain relatively bulky and are not aimed at individual physicians.

### SUMMARY OF THE INVENTION

In one form of the invention, it may be said to lie in a handheld medical diagnostic device including a display and processing unit; at least one probe unit adapted to produce medical diagnostic data; an interface adapted to connect a chosen one of a plurality of said probe units, each having a different medical diagnostic function and in general requiring differing communication and control protocols to be implemented in order to communicate with the display and processing unit, to said display and processing unit; the interface being configurable in use such that the chosen probe unit can be connected to the display and processing unit without user action to configure the interlace; the display and processing unit being adapted to receive the diagnostic data from the connected probe unit and to process, and analyse and display said data in a manner suitable for the nature of the diagnostic data.

In preference the display and processing unit is of substantially the same size and weight as the probe unit; and a physical layer of the interface includes a transmission cable of sufficient length to provide a means to locate the device about the neck of a user.

In preference the interface includes at least one diagnostic data connection for carrying the diagnostic data from the probe unit to the display and processing unit said data connection being adapted such that at least one of data transmission speed and data transmission protocol are able to be configured in use.

In preference the interface further includes a control data connection of fixed speed and protocol adapted to communicate information as to the data transmission speed and data transmission protocol required by the diagnostic data connection from the probe unit to the display and processing unit, to enable the diagnostic data connection to be configured when the probe unit is connected.

In a further form the invention may be said to lie in a probe unit having a diagnostic function for use with a display and processing unit including a sensor adapted to collect medical diagnostic data; an interface adapted to removably connect the probe unit to a display and processing unit; data storage to store data adapted to be communicated to the display and processing unit to identify the probe unit and its diagnostic function to the display and processing unit; the interface including a first data connection of fixed speed and protocol adapted to communicate with the display and processing unit.

In preference the interface further includes at least one diagnostic data connection for carrying the diagnostic data from the probe unit to the display and processing unit said data connection being adapted such that at least one of data transmission speed and data transmission protocol are able to be configured in use.

In preference the first data connection is a control data connection of fixed speed and protocol adapted to communicate information as to the data transmission speed and data transmission protocol required by the diagnostic data connection from the probe unit to the display and processing unit, to enable the diagnostic data connection to be configured when the probe unit is connected.

### BRIEF DESCRIPTION OF THE DRAWINGS

A preferred embodiment of the invention will now be described, by way of example only, with reference to the accompanying drawings in which:

FIG. 1 illustrates a preferred embodiment of a medical diagnostic device of the invention;

FIG. 2 is an illustration of the embodiment of FIG. 1 in use;

FIG. 3 is an illustration of the embodiment on FIG. 1 being carried by a user;

FIG. 4 illustrates a further embodiment of the invention, showing multiple, pluggable probe units;

FIG. 5 is a schematic block diagram of one form of implementation of the DPU of a preferred embodiment;

FIG. 6 illustrates an embodiment of a probe unit being an image based capture device;

FIG. 7 illustrates a schematic block diagram of the embodiment of FIG. 6;

FIG. 8 illustrates a simplified schematic block diagram of an ultrasound scanner diagnostic probe.

### DESCRIPTION OF PREFERRED AND OTHER EMBODIMENTS

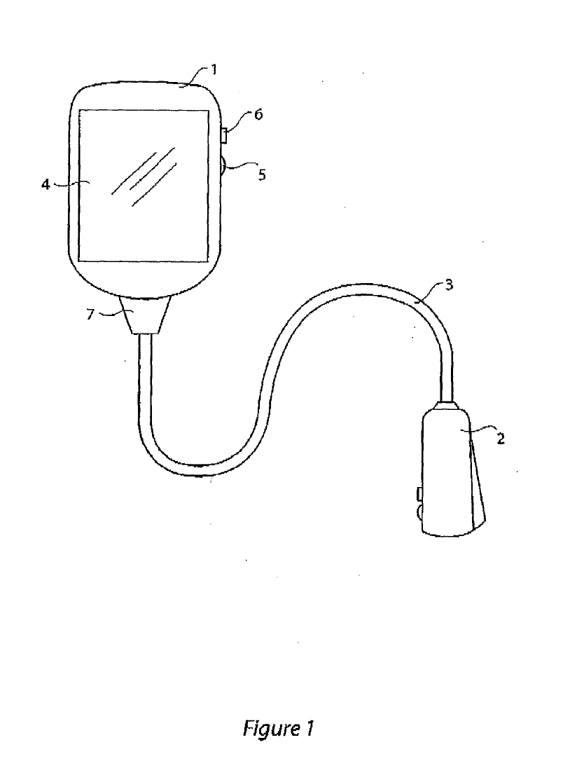

Referring to FIG. 1 there is illustrated a portable diagnostic device to be used by physicians at the bedside. There is a handheld display and processing unit (DPU) 1 connected to a diagnostic probe unit 2 via a cable 3. The cable attaches to the DPU via a plug and socket arrangement 7. In other embodiments, the plug and socket may be at the probe unit end of the cable, or may be provided at each end of the cable.

A variety of diagnostic probe units incorporating different types of sensors providing one or more diagnostic functions can be attached to the DPU. The DPU provides a configurable (programmable) interface, where the interface configuration is provided by the probe unit upon connection. The DPU does not need any user intervention to identify the requirements of a probe unit when it is plugged into the DPU. The interface provides a configurable data interface and may also supply power and an optical input interface.

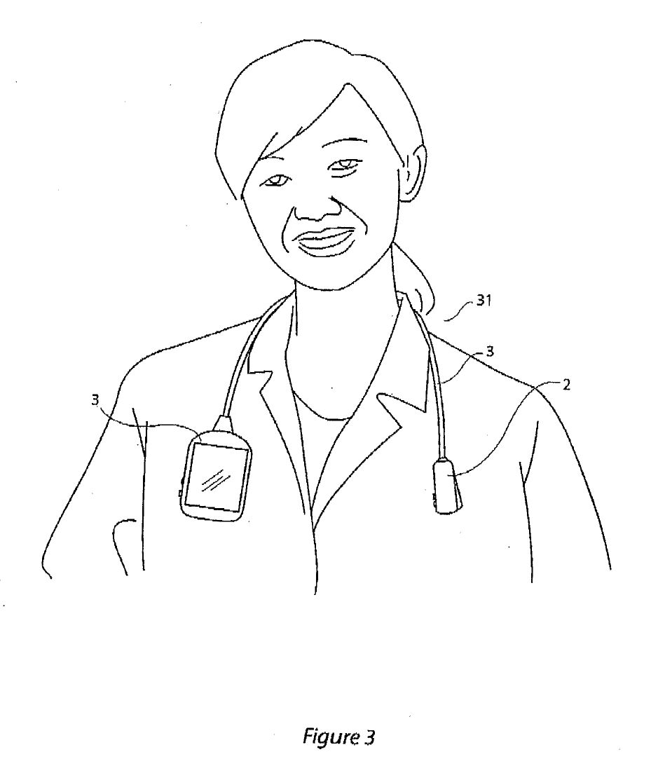

The handheld display and processing unit 1 and diagnostic probe unit 2 are designed to be of substantially equivalent mass, enabling the system to be conveniently stored around a user's neck, enhancing the portability of the device. An example of a user 31 implementing this mode of carriage is illustrated in FIG. 3.

The diagnostic probe adapts the system to any suitable diagnostic function. This function may be, without limitation that of audio devices, ultrasound scanners, otoscopes, ophthalmoscopes, blood testing devices, endoscopes, electro cardiogram devices, skin lesion testing devices, and vital signs testing devices.

The DPU includes a miniature colour display 4, such as a 320×320 pixel 65 k colour PDA) type display, or an 880×230 pixel digital camera type display. Any display which is small enough to fit into the DPU may be used.

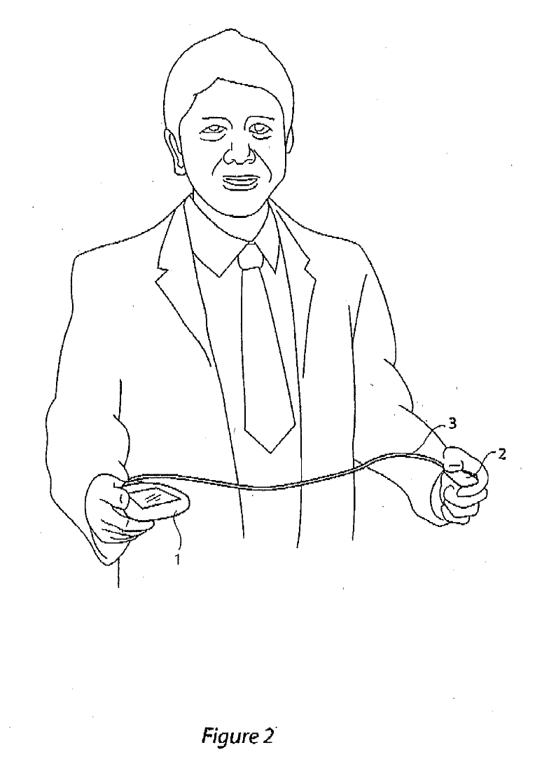

As shown in FIG. 2, the DPU 1 is of a size and shape to fit comfortably into a physician's or other user's 22 hand, with the diagnostic probe unit 2 being of a configuration to be readily applied to a patient with the other hand.

A variety of user input apparatus are provided. The handheld display and processing unit 1 provides a scroll wheel 5 and a button 6 for user input to allow control of most operations.

As illustrated in FIG. 2, the user input apparatus 5,6 can be operated by a user's thumb or finger when the DPU 1 is comfortably resting in the user's hand, freeing the second hand to hold and control the diagnostic probe 2.

In further embodiments, the screen 4 may be a touch sensitive screen, allowing user input with or without a stylus. A Bluetooth interface may be provided enabling the use of wireless keyboards or input devices. A microphone in conjunction with a dictation processing application may be provided for use for voice recording.

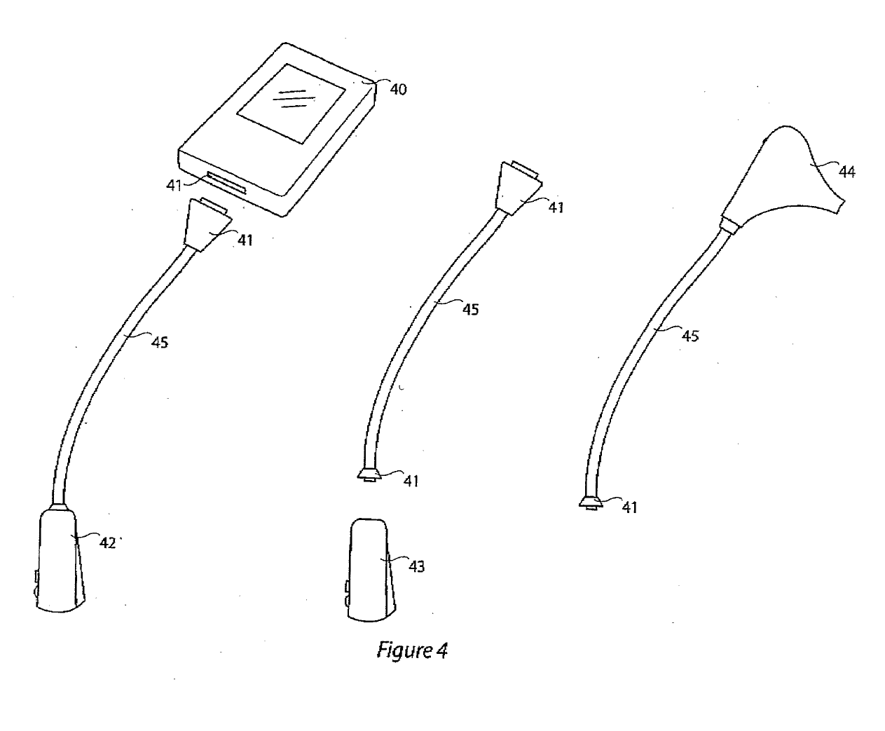

FIG. 4 shows a further embodiment of the device, with multiple pluggable diagnostic probe units. There is a DPU 40 and a series of diagnostic probe units: an audio auscultation sensor 42, an ultrasound scanner 43, and an optical image sensor 44. Each of the diagnostic probe units may be individually connected to the DPU by cable 45 and plug 41.

The cable 45 may be permanently connected to the probe unit as for the auscultation probe 42 or the cable may have plugs at each end as shown for the ultrasound probe 43.

Upon connection of any one of the probe units, the DPU automatically reconfigures the interface to the probe unit to provide the required communication protocol for communication with the probe unit, and runs software to provide the appropriate display and control features for the functionality of the connected probe unit.

The interface between the DPU and the probe units can support a variety of probe unit with different sensors and functions. The interface provides an always on connection between the DPU and the probe unit to read system identification and configuration information, enabling the DPU to always ensure the interface programmable logic device is configured correctly. On power up, or at first connection of a probe unit, the DPU will read the configuration PLD identification and read the probe unit identification. If they do not match, the DPU will read a new configuration from the probe unit, and program it into the programmable logic device (PLD) or field programmable gate array (FPGA), thus configuring itself to meet the requirements of the probe unit.

The interface can be implemented in a programmable logic device (PLD) or field programmable gate array (FPGA), with multiple physical layer interface integrated circuits. An embodiment of the invention could contain RS232, LVDS, USB1.1, USB2.0, and optical connections. The configurability of the interface allows for different probes to configure the interface for different specifications. For example, an audio probe may configure a data communications channel of the interface to use the unidirectional I²S serial format suited for audio interface rates (64 kbits/sec to 5 Mbits/sec), while an ultrasound probe may configure the data channel to use a high speed serial protocol (>20 Mbits/sec).

The programmable device enables the system to use the microprocessor's native interfaces, enabling sensor data to be written directly into the microprocessor's internal or external memory without intervention from the microprocessor's processing unit, minimising power consumption. The same configurable interface can be configured to connect to a non-sensor device, such as a personal computer utilising on-the-go USB protocols.

An optical based device such as an otoscope could use the optical interface to direct the received optics to a camera sensor. By providing a camera sensor on the DPU, the overall system cost is reduced when supporting several optical sensors, such as otoscopes, ophthalmoscopes, and endoscopes.

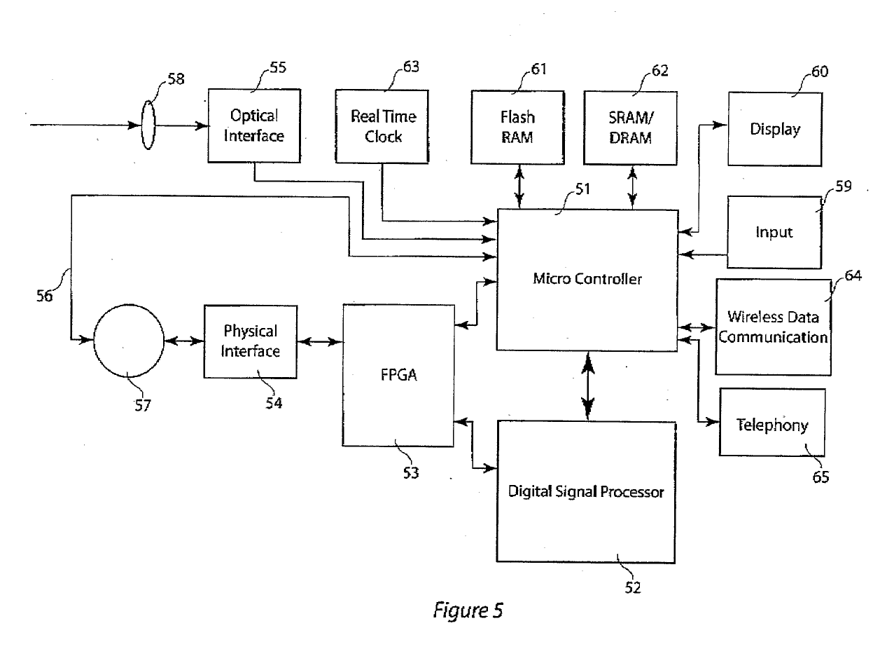

A schematic arrangement of the technical components of an embodiment of the device can be as illustrated in FIG. 5. The illustrated arrangement provides a functional diagram of the DPU component only. It will be evident to the skilled hardware designer that the preferred embodiment can be implemented in many different electronic forms.

The forms can include standard microcontroller and DSP/FPGA components, or a full custom ASIC design may be employed. Hence, the system could be constructed of numerous separate components (such as op-amps, A/D converters, D/A converters, digital signal processors, memory, displays, communications components etc), or could be comprised primarily of a mixed-mode application specific integrated circuit (ASIC) with a small number of support components. An ASIC would provide cost, power consumption, and size advantages.

Referring to FIG. 5, there is a microcontroller 51 and digital signal processor 52. A field programmable gate array 53 provides the configurable logical interface to probe units. This is connected to the physical layer interface components 54. An optical interface 55 and an optical aperture 57 are provided for direct optical connection to probe units having an optical sensor capability.

An always-on channel 56 is provided for communication of interface configuration data from a probe unit to the DPU.

User input hardware 59 is provided, which may include any or all of a keypad, a scroll wheel, a push button and a navigator button. An output device in the form of a display 60 is also provided.

The microcontroller 51 controls user input and output. The dedicated DSP (or DSPs) provides faster digital signal processing. Devices such as the Texas Instruments OMAP, Intel PCA series, or Motorola IMXC contain both power efficient microcontrollers and DSP, and therefore would be suitable for use. Memory for program execution and firmware storage are provided as non-volatile memory 61 and volatile RAM 62.

The device may read firmware specific to a particular probe unit from the probe unit at power on or at the connection of the probe unit. The storing of firmware in the probe allows any new probe to operate with the DPU without the DPU having to be configured by the user. Alternatively, the DPU could read the probes unique identification using the always on connection 56, and download the configuration and firmware automatically from an internet connection.

A real time clock 63 is provided for keeping time. Wireless communications unit 64, which may conform to the Bluetooth, 802.11 or any other convenient standard, is included to provide communications to computer networks, or to local devices such as headphones.

Cellular telephony communications 65 can be provided to provide voice communications to another cellular telephony user or to provide data access to the internet or another computer network.

A wired communications system such as USB 2.0 or firewire (IEEE1394) may also be included. Using these communications systems, the user can save or download recorded patient data to an alternative system, such as but not limited to a medical records database operating on a personal computer, network server, or mainframe computer.

In different embodiments, the probe unit to DPU interface can utilise one or more physical interfaces, which may be USB1.1, USB2.0, Firewire, LVDS, RS232, optical, or any other suitable physical interface.

An embodiment of the invention incorporates a secure data (SD) slot, enabling users to insert non-volatile flash memory cards. Another embodiment could incorporate a miniature hard disk in the DPU. The user interface can be manipulated such that all measurements taken by the device are recorded to non-volatile memory, along with a timestamp and other data identifying the patient.

The device of the invention provides the advantage that a user/physician need only carry a single device with a small number of optional probes in order to have available a significant range of sophisticated diagnostic devices for everyday use.

Probe units may include any functionality which might find it advantageous to have readily available, which can be provided by electronic or optical or acoustic means.

An embodiment of a probe unit with one or more audio sensors can provide electronic stethoscope or auscultation functionality. Audio output is provided by the DPU by an encapsulated speaker; a set of headphones connected by wire; or a set of wireless headphones connected via a wireless protocol such as Bluetooth, or any other convenient means. The audio signal can also be processed and a visual representation output via the DPU display, with either filtered envelope detection plots, colour spectral plots, or frequency plots or any other desired result of applying signal processing to the audio signal.

The DPU can also be configured with software to analyse the incoming audio signals and to provide automated diagnosis or at least diagnostic assistance. For example, the system can be configured for heart sound diagnosis, where the DSP processes the input signal looking for information consistent with known heart conditions, such as murmurs and abnormal heart sounds. The DPU architecture allows different algorithms to be developed and implemented by in use changes to the DPU firmware. This may be by means of separate download from a network to the DPU, or the firmware upgrade may be provided by a probe unit.

In one embodiment the DPU implements an algorithm for a user controlled calibration procedure, to compensate for hearing loss in physicians. The result of the calibration process is a map of the user's hearing profile. In general a user's dynamic range for hearing will be different for different frequencies. The DPU is able to compensate for the varying dynamic range of the user's hearing by applying frequency dependent enhancement of the audio signal

A pressure sensor can be included in the audio probe unit to enable pulse detection. The filtered pressure sensor is converted to an audio output signal by modulating with an audio noise signal. The pulse at the extremities of a patient's limb can be detected to diagnose the possibility of blockages of arteries. Another method is to use the audio input signal and to process the signal using wavelets derived from typical pulse shapes.

The pressure sensor can also be used to replicate the sound of traditional stethoscopes. Popular stethoscopes (such as the Littman series) are known to have a distinctive frequency response. U.S. Pat. No. 6,026,170, and U.S. Pat. No. 6,134,331 describe the use of electronic means to replicate the frequency response of popular stethoscopes. However, in some stethoscopes, for example the popularly used Littman stethoscope, the frequency, response changes according to the downward pressure applied by the stethoscope user. An embodiment of an audio probe unit can overcome this limitation by detecting the downward pressure applied by the user using a physical pressure sensor mounted on the transducer, and digitally adjusting the response to replicate the response of the desired stethoscope.

To detect sub-dermal structures physicians commonly use percussion. An embodiment of an audio probe unit provides an automated percussion apparatus wherein an audio speaker transmits an impulse, then microphones capture the resonant signal. The resonant signal is converted to a digital signal and transmitted to the DPU, where the signal corresponding to the generated pulse is removed. The signal is filtered and amplified before being converted to audio by the output speaker or headphones. The spectral response can also be drawn on the display. The resonant sound provides a physician with an indication of the sub-dermal structure below the probe.

The utility of percussion can be improved by the inclusion of a measuring device. The measuring device can be used in conjunction with the percussor to record the size of imaged structures. The user locates a structure boundary using the percussor mode, and initiates a measure by pressing a button (or some other means such as a voice command). The user then locates the other boundary of the imaged structure, and releases the button, presses another button, or issues another voice command. The device records and displays the distance between the two boundaries. A number of techniques can be used for calculating the distance including accelerometers, rotary encoders, or any other method suitable for measuring position.

Image capture probe units may be used to provide the functionality of any single function instrument which allows the user to visually see a feature of interest, whether directly of by a camera. Examples of such instruments are otoscope devices for ears, ophthalmoscope devices for eyes, laryngoscopes for the throat, and endoscope devices for inner body imaging.

Such probe units provide a means of detecting an image of a region, and either transferring the image to the DPU via a fibre optic cable or detecting the image in the probe and transmitting image data over a data connection provided by the interface.

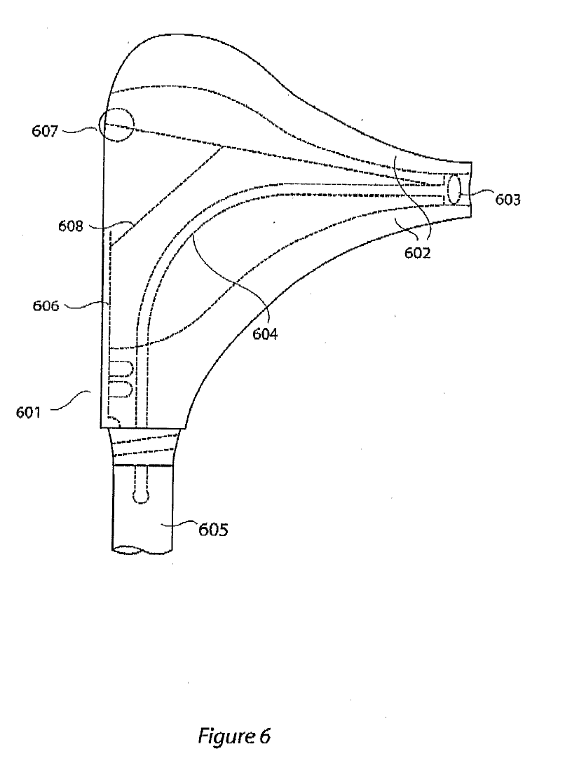

An embodiment of an image probe unit in the form of an otoscope is illustrated in FIG. 6. There is a light source 601 in the form of white light emitting diodes. A light cavity 602 directs the light to the area of interest. A lens system 603 focuses the reflected light to an optical fibre 604. The optical fibre transmit the light via interface cable 605 to the DPU (not illustrated). Interface electronics 606 are provided to allow communication between the probe unit and the DPU.

Electronic focus control 608 allows for control of focus from the DPU, or automatic focus by electronics in the probe unit. A manual focus control 607 is also provided.

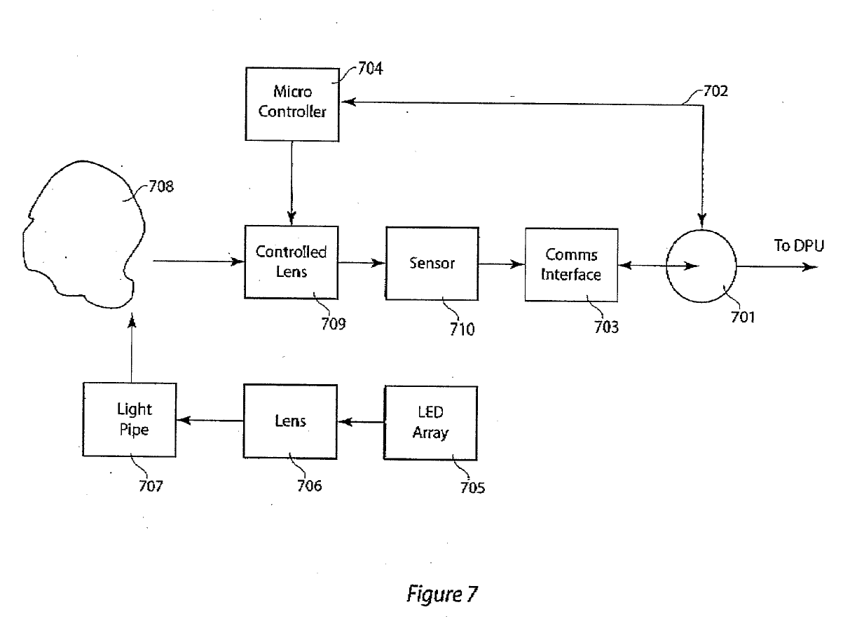

A block diagram representation of a general image capture probe unit is shown in FIG. 7. In this embodiment, there is an interface connector 701 providing connection to a DPU (not shown) for a low speed, low power, always on data connection 702 and a high speed data connection 703, which is active only when transfer of diagnostic sensor data to the DPU is required.

There is a microcontroller 704 which runs firmware for control of the instrument and provides a control interface for the user of the DPU to control user controllable functions of the probe unit.

There is a LED array 705 which provides a light source. This is directed to the area to be visualised 708 by a lens system 706 and a light pipe 707. Light reflected from the area to be visualised is collected by a lens and lens control system 709. The lens control system is controlled by the microcontroller to focus light on an image sensor 710. The focussing requires the movement of a lens, a sensor, or both and can be achieved using a motor, mechanical means, or MEMS. This focus control may be automatic, or controlled by the user from the DPU, or controlled by the user locally at the probe unit.

The image sensor converts the light image to a data stream which is communicated to the DPU via the high speed data connection 703.

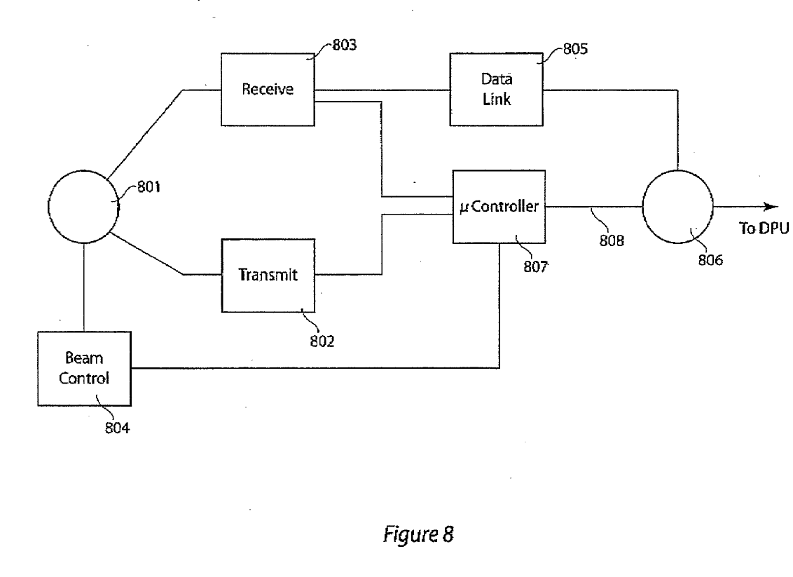

A further embodiment of a probe unit is illustrated as a block diagram in FIG. 8. This probe unit allows the system to provide the functionality of an ultrasound scanner. The probe unit contains circuitry for generating, transmitting, and capturing ultrasound signals.

There is a transducer 801 which is adapted to transmit ultrasound energy into an area of interest of a subject's body in response to an electrical excitation from the transmit electronics 802, and to receive echoes from the subject's body, which are converted to electrical data by the receive electronics 803. In order for an area of the body to be imaged, the ultrasound beam must be swept over the scan area. This is accomplished by the beam directional control apparatus 804, which physically moves the transducer. This may be in the form of a stepper motor, or any other convenient mechanical arrangement. In an alternative embodiment, the transducer may be an array of transducer elements, and the scanning beam may be formed electronically by selective activation of transducer elements by the transmit electronics.

The data stream from the receive electronics is transmitted to the DPU (not shown) by the high speed data link 805, through the physical connector 806. This high speed data link is only operational when it is necessary to transmit data to the DPU.

The probe unit is controlled by a microcontroller 807. The microcontroller also maintains an always on data link 808 for communication with the DPU. This data link allows the probe unit to communicate to the DPU using little power to cause the DPU to configure the high speed connection with appropriate parameters for communication with the probe unit.

The DPU can be configured to process the ultrasound using several means available through the background art. The ultrasound can be converted to grey scale and displayed on the local display, processed for Doppler, down sampled, and sent to one of the audio outputs, or processed for Doppler and a colour display overlaid on the grey scale display.

The utilisation of a MEMS base ultrasound probe increases the utility of the probe by incorporating different transducer types in the same probe. A linear probe utilises transducers designed to operate at higher frequencies and is suitable for surface imaging. This allows the probe to be used as a cannula insertion aid. On the same device, a phased array probe would use transducers at a lower frequency, suitable for deeper imaging.

Other embodiments of probe units may be used to allow the system to provide additional functionality.

A probe unit may include a laser scanner for interfacing to analysis devices, or colour sensitive skin patches.

Probe units may include ultrasound sonoporous functionality, whereby ultrasound is driven into a patient's skin thereby opening fluid transmission channels.

Probe units may include spectrometers, biochips, or any other electronic means for providing blood testing functionality.

Probe units may include devices for analysing electrical activity associated with nerve impulses to provide electroencephalogram (EEG) functionality

Probe units may include apparatus to allow the system to provide the functionality of a dermatoscope which is used for analysing skin lesions.

Probe units may include apparatus to allow the system to provide the functionality for measuring a range of vital signs such as blood pressure, pulse, and oxygen saturation.

Alone or in combination with diagnostic functionality, probe units may include therapeutic attachments such as devices facilitating fluid removal or ear wax removal.

Probe units may include any circuitry that can provide a useful diagnostic or therapeutic functionality.

Although the invention has been herein shown and described in what is conceived to be the most practical and preferred embodiment, it is recognised that departures can be made within the scope of the invention, which is not to be limited to the details described herein but is to be accorded the full scope of the appended claims so as to embrace any and all equivalent devices and apparatus.

1-41. (canceled)

42. A handheld medical diagnostic device including
a display and processing unit;
at least one probe unit which produces as an output medical diagnostic data the display and processing unit operating to receive the diagnostic data from the probe unit, the probe unit being of a type selected from a plurality of probe unit types each of which provides a different type of diagnostic data to allow the device to fulfil a different medical diagnostic function,
an interface which removably connects said probe unit to said display and processing unit;
wherein upon connection of a probe unit to the display and processing unit identification and specification data is communicated from the probe unit to the display and processing device indicating the nature and function of the diagnostic data output by the probe unit;
the display and processing unit then running programs to process, and analyse and display said diagnostic data in a manner suitable for the nature of the diagnostic data.

43. The device of claim 42 wherein the probe unit is of a type selected from a plurality of probe unit types each of which requires specified communication and control protocols to be implemented in order to communicate with the display and processing unit,
wherein the interface is configurable in use such that when a chosen probe unit is connected to the display and processing unit via the interface, the required communication and control protocols for the chosen probe unit are implemented without user action. to configure the interface.

44. The device of claim 42 wherein the display and processing unit is of substantially the same size and weight as the probe unit;
and the interface includes a transmission cable of sufficient length to provide a means to locate the device about the neck of a user.

45. The device of claim 43 wherein the interface includes at least one diagnostic data channel for carrying the diagnostic data from the probe unit to the display and processing unit said data channel being configurable such that at least one of data transmission speed and data transmission protocol are able to be configured in use.

46. The device of claim 45 wherein the interface further includes a control data channel of fixed speed and protocol which communicates information as to the data transmission speed and data transmission protocol required by the diagnostic data channel from the probe unit to the display and processing unit, to enable the diagnostic data connection to be configured when the probe unit is connected.

47. The device of claim 42 wherein the interface includes a Universal Serial Bus (USB) link which carries the diagnostic data from the probe unit to the display and processing unit.

48. The device of claim 42 wherein the display and processing unit includes an image sensor and a fibre optic connection adapted to carry visual images from a probe unit to the image sensor located at the display and processing unit.

49. The device of claim 48 wherein the image sensor is a charge couple device (CCD) sensor

50. The device of claim 42 wherein the display and processing unit includes a user interface through which a user may control at least some functions of the display and processing unit and of the probe unit.

51. The device of claim 42 wherein the display and processing unit includes wireless communications apparatus for connection to a computer network or a telecommunications network.

52. A probe unit which produces as an output medical diagnostic data for use with a display and processing unit including a sensor which collects data having medical diagnostic information;
an interface to removably connect the probe unit to the display and processing unit, the interface including a first communication channel able to carry data communications between the probe unit and the display and processing unit;
and data storage to store data to be communicated to the display and processing unit to identify the probe unit and its diagnostic function to the display and processing unit.

53. The probe unit of claim 52 wherein the interface further includes at least one diagnostic data channel for carrying the diagnostic data from the probe unit to the display and processing unit said diagnostic data channel being such that at least one of data transmission speed and data transmission protocol used by the data channel are able to be configured in use.

54. The probe unit of claim 53 wherein the first communication channel is a control data channel of fixed speed and protocol adapted to communicate information as to the data transmission speed and data transmission protocol required by the diagnostic data channel from the probe unit to the display and processing unit, to enable the diagnostic data channel to be configured when the probe unit is connected.

55. The probe unit of claim 52 wherein the first communication channel is implemented via a USB connection, said channel carrying control and diagnostic data between the probe unit and the display and processing unit.

56. The probe unit of claim 52 wherein the sensor is an audio input sensor and the probe unit further includes a surface pressure sensor able to sense the degree of pressure being applied by the probe unit to a patient's body, the display and processing unit including computer programming to process data from the surface pressure sensor and to adjust the audio input received from the audio input sensor to match the response of any one of a number of pneumatic type stethoscopes.

57. The device of claim 42 wherein the probe unit includes an audio input sensor and an audio output device and the display and processing unit is adapted to initiate the audio output device to generate an audio impulse, and the audio input sensor receives a resultant signal.

58. The device of claim 57 wherein the display and processing unit processes the resultant signal to remove the impulse output, filter the signal, amplify the signal, and transmit the signal to an audio output.

59. The device of claim 58 wherein the display and processing unit converts the signal to the frequency domain, and displays the output on a display of the display and processing unit.

60. The device of claim 57 wherein the probe unit further includes a position measurement sensor, adapted to send position data relating to the position of the probe unit to the display and processing unit, the display and processing unit processing the position data and the resultant signal to produce a display plotting a characteristic of the resultant signal against relative position of the probe unit.

61. A probe as claimed in claim 60 where the measurement sensor is an accelerometer.

62. A probe as claimed in claim 61 where the accelerometer is a MEMS based accelerometer.

63. The device of claim 42 wherein the probe unit includes an image sensor which receives a light image and converts the image to a data stream, the data stream being communicated to the display and processing unit.

64. The device of claim 63 wherein the probe unit provides functionality selected from the functions of an otoscope, an ophthalmoscope, a laryngoscope, sigmoidoscope, and a colonoscope.

65. The device of claim 42 wherein the probe unit includes at least one ultrasound transmitting and receiving transducer and the diagnostic data produced is ultrasound scanlines; the display and processing unit, when connected to the probe unit, running programs to produce and display ultrasound images from the diagnostic data.

66. The device of claim 42 wherein the probe unit includes a laser scanner for providing laser scanned measurements to the display and processing unit.

67. The device of claim 42 wherein the probe unit includes an ultrasound generator for opening fluid pathways in dermal structures.

68. The device of claim 42 wherein the probe unit includes a spectrometer for collecting spectral information on fluid samples and transmitting the spectral information to the display and processing unit for processing.

69. The device of claim 42 wherein the probe unit includes a biochip for testing fluid samples, and transmitting biochip data to the display and processing unit for further processing.
